(19) United States
(12) Patent Application Publication
(10) Pub. No.: US 2016/0166184 A1
(43) Pub. Date: Jun. 16, 2016

(54) MICRONEEDLES AND METHOD OF MANUFACTURE

(71) Applicant: SWANSEA UNIVERSITY, Swansea (GB)

(72) Inventors: Kar Seng Teng, Swansea (GB); Jonathan Lloyd, Swansea (GB)

(21) Appl. No.: 14/905,455

(22) PCT Filed: Jun. 14, 2014
(86) PCT No.: PCT/GB2014/000287

§ 371 Date: Jan. 15, 2016

(30) Foreign Application Priority Data
Jul. 16, 2013 (GB) 1312669.3

(51) Int. Cl. A61B 5/145; A61B 5/1473

(52) U.S. Cl. CPC A61B 5/14514; A61B 5/14532; A61B 5/14735; A61B 5/14546; A61B 2562/0285; A61B 2562/125

(57) ABSTRACT

A microneedle device (200) including at least one microneedle (1) having one or more nanowires (203) on a surface of said at least one microneedle. The microneedle device is typically used in a sensor such as a sensor for monitoring glucose levels in the body and the nanowires may have a membrane (207) covering at least part of the nanowires.

### FIELD OF THE INVENTION

The present invention relates to microneedles and a method of manufacture. Furthermore the invention relates to a biological fluid monitoring device including said microneedles and in particular but not exclusively to a sensor device that is minimally invasive and which in particular monitors the glucose level of an individual.

### BACKGROUND OF THE INVENTION

The most common glucose measurement available today is the ‘finger stick’ glucose meter. Diabetic patients would normally perform the test between 3 to 10 times a day, which is painful and causing the finger to sore. Due to discomfort, the non-compliance with a finger stick test is partly responsible for the increase in the uncontrolled condition. Furthermore, such tests only provide a discrete observation of glucose level that changes continuously with time, thus it cannot allow the patient to monitor how the glucose level reacts with insulin, exercise, food and other factors. Therefore, a continuous glucose monitoring (CGM) system is critical for the adjustment of correct insulin dosing ratios for food intake and correction of hyperglycemia. An effective CGM system will significantly reduce the risk of health complications associated with diabetes and therefore enhancing the patients' quality of life and their life expectancy.

More recently, individuals can take their own glucose measurement using special instruments, for example in people with diabetes. The special instruments may include microneedles which have the advantage that they may be applied to the body via the skin without pain and bleeding. Microneedles are sufficiently long enough to penetrate through the stratum corneum skin layer and into the epidermal skin layer but also are sufficiently short not to penetrate the dermal layer. Known devices may be used to take individual samples or they may form part of a device that can be used to sample and filter fluids within the skin tissue on a continual basis. An example of a device using microneedles can be found in US 2009/0043250.

Known devices have limitations, in that they are used to measure very small amounts of substances in the body but they may not be sensitive enough to accurately distinguish some analytes from others in the body with the required level to give an accurate measurement for an accurate diagnosis of the condition of the individual. In the case of glucose for example, the glucose level in the body is less than one thousandth that of other chemicals in the body and so there is a need for increased sensitivity of monitoring or measuring devices.

The present invention seeks to overcome the problems associated with the prior art by providing an accurate way of measuring chemicals in the body and in particular the invention provides a continual way of monitoring biological fluid, with minimal impact on the individual that is being monitored. Furthermore, the invention allows for real time evaluation of biological fluids in a minimally-invasive, painless, and convenient manner and with a high degree of sensitivity for materials within the body. In addition the present invention overcomes the problems of having blocked needles and difficulties in fluid extraction that are associated with known devices.

### SUMMARY OF THE INVENTION

According to a first aspect of the present invention there is provided a microneedle device for a sensor including at least one microneedle having one or more nanowires on a surface of said at least one microneedle. Typically the microneedle does not have a bore.

It is envisaged that the microneedle device has a plurality of said microneedles formed as an array on a support surface, each microneedles having one or more nanowires on the surface thereof. Typically the microneedles are arranged in alignment with one another so there is a regular array of microneedles on the support surface.

Preferably, the at least one microneedle having one or more nanowires on a surface of said microneedles has a membrane disposed there over as to at least partially cover the one or more nanowires.

It is envisaged that the microneedles and/or the nanowires are functionalized for the detection of an analyte.

It is envisaged that the membrane is an ion selective membrane. The ion selective membrane is arranged such that one or more analytes can pass through the membrane. Typically the membrane is a porous polymeric membrane and the pores may be selected for filtering out particles or molecules in the biological fluid that are above a certain size.

Preferably the nanowires are formed of Zinc Oxide.

According to a second aspect of the invention there is provided a device for the monitoring of biological fluid from an individual, said device comprising an array of microneedles, characterized that one or more of said microneedles includes one or more nanowires on a surface of said microneedles, said one or more of said microneedles and/or nanowires having been functionalized with material that can interact with a material in the biological fluid of an individual that contacts said microneedles so said device can monitor one or more anayltes in the biological fluid. The biological fluid typically is interstitial fluid in the skin.

It is envisaged that there is a membrane disposed to lie over said one or more microneedles, said membrane being porous so that fluid can pass through the membrane and come into contact with the nanowires and microneedles.

It is preferred that the microneedles/nanowires are in two or more arrays with each array being functionalized with different material from another array.

Preferably the microneedles having nanowires thereon act as a working electrode for the device which is a sensor for analytes in the biological fluid of an individual.

Preferably the microneedles on which the nanowires have been formed have a membrane disposed thereon so as to at least partially cover the one or more nanowires.

It is envisaged that the biological fluid is interstitial fluid. Typically interstitial fluid is the main component of the extracellular fluid in the body that bathes the cells of the body.

Preferably at least one of the analytes in the biological fluid is glucose.

Typically the sensor has two electrodes, a working and a reference electrode. Alternatively the sensor has three electrodes, a working, counter and reference electrode. The needles are divided into two or three groups according to whether there are two or three electrodes.

It is envisaged that the support structure be coated with an insulating material to isolate areas which are not microneedles from analytes.

It is envisaged that the device is releasably securable to a control system, said control system including controls for the sensor and a transmitter.

Preferably the control system includes an accelerometer attached thereto.

It is envisaged that data from the transmitter or accelerometer is transmitted using wireless communication, preferably the transmission being in real time although data can be stored and transmitted at another point in time.

It is further envisaged that the control system includes a temperature sensor to monitor the body temperature of a wearer of the device. The sensor reading can be adjusted to compensate for the effect of temperature on the sensor output. In addition though according to the temperature detected by the device then drug delivery can be altered accordingly as the temperature can be an indication of the metabolism of the individual and drugs may have to be delivered at a rate to suit an individual's metabolic rate.

According to a further aspect of the invention there is provided a monitoring system provided by a device according to the second embodiment of the invention in combination with a control system including controls for the sensor, a transmitter and an accelerometer.

Preferably the monitoring system includes a receptor on the nanostructures, said monitoring system being arranged to monitor heart diseases, stroke, chronic obstructive pulmonary diseases or asthma.

It is envisaged that the technology is particularly useful to measure diabetes but it can be used for monitoring other chronic diseases, such as heart diseases, stroke, chronic obstructive pulmonary diseases, asthma and cancers etc., through the use of an appropriate receptor at the surface of the nanostructures formed of the microneedles and nanowires.

According to a further aspect of the invention there is provided a method of fabricating nanowires onto microneedles, said method including forming a seed layer onto the microneedles and rotating said microneedles so that a centrifugal force is formed that draws the spray of material along the length of the microneedles to coat the surface of the microneedles following which the nanowires are formed from the seed layer.

Preferably the seed layer is formed on the microneedles by spraying.

It is envisaged that the seed layer is subsequently annealed to initiate nanowire production by growth of said nanowires grown from the surface of a microneedle.

As an alternative the seed layer is formed on the microneedles by plasma vapor deposition (PVD).

Where the seed layer is deposited by plasma vapour deposition (PVD) on the microneedles the sample is rotated at an angle to distribute the seed layer evenly all around the microneedles. In this production process the seed layer does not need to be annealed. It is desirable that the sample is not perpendicular to the incoming material. Samples are usually rotated in PVD for a more even distribution but in the current invention the rotation is at an angle to get the material to deposit on the sides of the features. PVD normally will not deposit onto the side of vertical features and this is why the angled rotation is required.

Preferably the material to form the seed layer is zinc acetate.

### BRIEF DESCRIPTION OF THE DRAWINGS

An embodiment of the invention will now be described by way of example only with reference to and as illustrated in the accompanying Figures in which:

FIG. 1 shows: a nanowire sensor device according to the prior art;

FIG. 2 shows: a schematic of the various stages in the production of microneedles according to an embodiment of the invention;

FIG. 3 shows: apparatus used in the manufacture of a microneedles/nanowire sensor according to an embodiment of the invention with FIG. 2a showing a plan view and 2b being a side view; and

FIG. 4 shows: a sensor including microneedles and nanowires according to an embodiment of the invention.

### DETAILED DESCRIPTION OF THE INVENTION

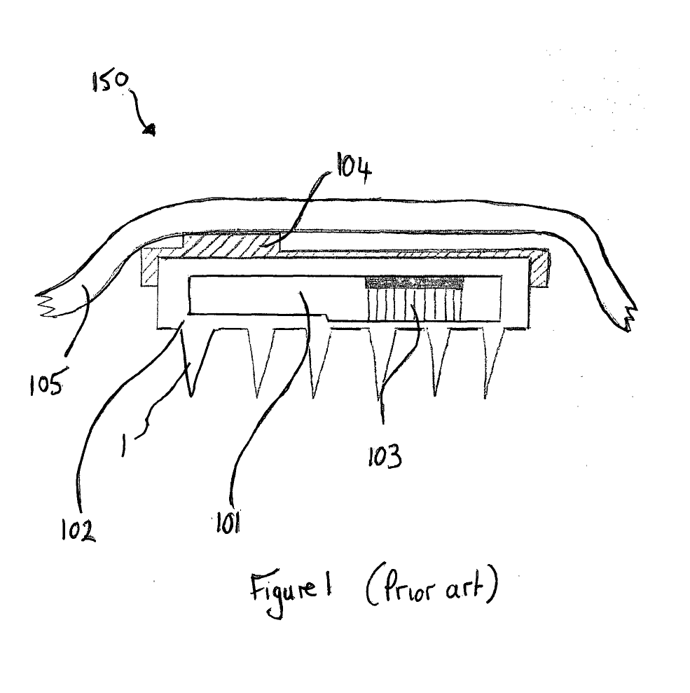

A prior art biological fluid monitoring device is shown generally as 150 in FIG. 1. The device including microneedles 1 that are used to take up biological fluid, such as interstitial fluid from an individual, a reservoir 101 for receiving the fluid from the body and a sensor 102 that is housed within the reservoir and which analyzes the biological fluid components and composition. The sensor has nanostructures 103 within the body of the sensor and which are associated with an electrode for the sensor and these nanostructures having large surface to volume ratio increases the sensitivity of the sensor as the biological fluid passes by when flowing through the reservoir. The nanostructures are grown from an electrode on an electrode support that can be attached to surface of the sensor and in effect forms part of the sensor. The nanostructures are preferably in the form of aligned wires as shown and are grown using hydrothermal technique from a seed layer on the electrodes that are positioned on the plastic support and typically the electrodes are printed onto this support.

The device itself forms a first part of a monitoring system and this first part is attached to a second part that is formed of the controls 104 which can collate and record data.

The microneedles 1 typically provide as an array that can come into contact with the body. The microneedles allow for the uptake of interstitial fluid from under the surface of the skin. The length of the needles will be sub in the region of 500 to 1500 μm, more particularly 700 to 1000 μm, meaning the device will be entirely painless as the microneedles are sized to avoid or minimize contact with nerve endings in the biological tissue, such as the dermis, thereby eliminating or reducing pain when the microneedles are inserted, for example into the skin. The microneedles are attached to a substrate 102 to which the base of the microneedle(s) is secured or integrated, and at least one reservoir/fluid collection chamber 101 and/or sensor 102 is in communication with the array of microneedles. Fluid can flow from the interstitial fluid by way of a bore in the microneedle that allows the fluid to flow into reservoir 101 where the sensors are situated.

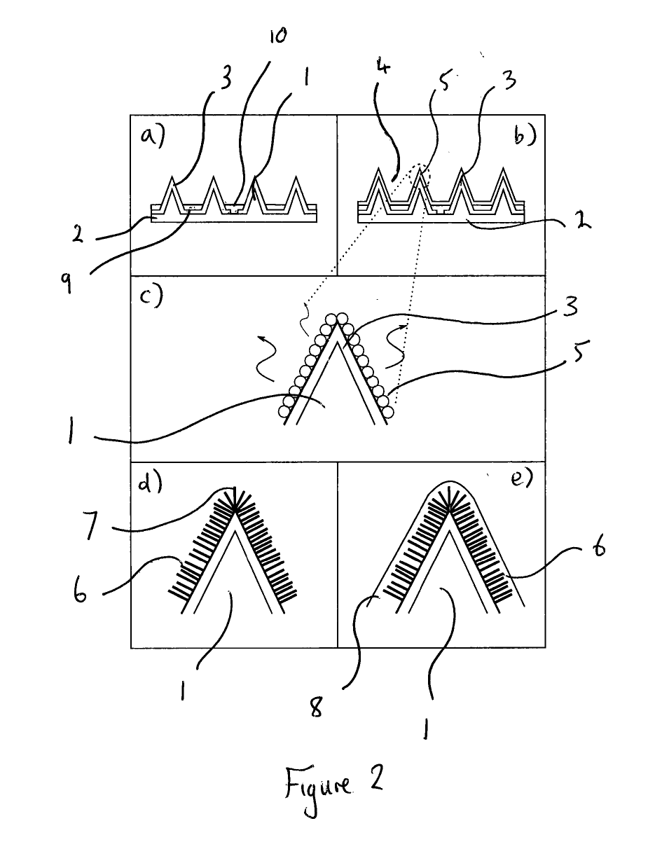

FIG. 2 shows a schematic of the production of microneedles that are to form a microneedle device according to an embodiment of the invention. The microneedles 1 are formed on a substrate 2 which may be constructed from a variety of materials including metals, ceramics, semiconductors, polymers or composites. The microneedles of the device can be constructed from a variety of materials, including metals, ceramics, semiconductors, organics, polymers, and composites. Preferred materials of construction include pharmaceutical grade stainless steel, gold, titanium, nickel, iron, tin, chromium, copper, palladium, platinum, alloys of these or other metals, silicon, silicon dioxide, and polymers. A preferred material that is used is a polyimide or SUB. The microneedles can be oriented perpendicular or at an angle to the substrate. Preferably, the microneedles are oriented perpendicular to the substrate to provide structural strength and to permit ease of insertion into the tissue. An array of microneedles can include a mixture of microneedle orientations, heights, spacings, or other parameters. This variation in an array can be useful, for example, if different microneedles are to provide different sensing or insertion functions. The substrate forms the base to which the microneedles are attached or integrally formed. The microneedles should have a mechanical strength to maintain intact while positioned in the skin of an individual. Unlike known microneedles, the microneedles of the present invention do not need a bore for the uptake of analyte from the body which is to be sampled, or through which a drug can be delivered to an individual. The fact that there is no bore means that making the microneedles can be much more cost effective because producing bores is technically difficult and hence costly to the manufacturing process.

An alternative process to producing microneedles using a seed layer is that where the needles are made from a polymer then the microneedles may be made by injection moulding.

The microneedles themselves again can be constructed from a variety of materials including metals, ceramics, polymers, composites. As shown in 2a) the microneedles are fabricated to a length of 700-1000 micrometers as this allows the microneedles to penetrate into the skin and on into the epidermis so that the nanowires that are on the microneedles can be used to monitor, for example glucose that is present in the interstitial fluid. The microneedles can be straight or as shown they may have a tapered shaft but generally where there is no tapering to an apex, the device is referred to as a microtube.

The microneedles are coated with a conductor such as gold or a carbon ink or indeed graphene 3 as shown in FIG. 2a, which is then segmented, usually through photolithography (FIG. 2b) so that troughs 4 are formed between the needles and these individual needles form the different electrodes that enable the sensor to operate. The needles are covered with the conductor and also an area between the needles that forms the needle support. The conductive material between the needles on the needle support is coated with an insulator, typically SU-8 through a photolithographic technique. This ensures that only the penetrating part of the needle is used for sensing the analyte. The microneedles form the working electrode and in a sensor there is also a reference electrode (not shown) which is usually electroplated with silver and chlorinated to form a silver/silver chloride reference electrode. There is an insulating layer 9 between the needles and this layer is used to isolate the needle support from the analyte in order to reduce interference from, for example perspiration. There are also gaps 10 between groups of needles to separate sets of electrodes.

FIG. 2a shows a similar arrangement to that in FIG. 2(a) except that the seed layer (which is shown more clearly in FIG. 2(c) is deposited over the conductive layer that forms the working electrode.

As shown in FIG. 2c) the microneedles are coated with a seed layer which can allow for hydrothermal or electrochemical growth of nanowires from the surface of the microneedles. Typically the seed layer is zinc acetate or PVD deposited nanocrystals that allows for the growth of nanowires made from Zinc Oxide (ZnO). It is important that the zinc acetate is deposited evenly on the 3D structure of the microneedles so that the microneedles can perform as efficiently as possible. The ZnO coated structures (nanowires or nanowires and microneedles) are typically formed by hydrothermal growth at 70 to 90 degrees centigrade. The growth solution is typically 25-30 mM Zinc Nitrate and Hexamethylenetetramine (HMTA) and this is grown for around 3 to 6 hours to form the nanowires. The production time of the nanowires can be further reduced by an electrochemical growth method. In this method the sample is immersed in a solution of zinc chloride and potassium chloride at concentrations of 1 mM to 10 mM and 0.05 M to 0.5 M respectively and heated to between 50° C. and 90° C. A counter electrode and Ag/AgCl reference electrode are also required in the solution and a potential of −0.8V to −1.2V is applied to the sample with respect to the reference electrode. After 500 to 3000 seconds suitable nanowires are grown.

FIG. 2d) shows a microneedle with nanowires 6 along the length of the microneedle. It is preferred that the nanowires are substantially parallel and extend roughly at right angles to the surface of the microneedle. However it is possible to have nanowires that cross over one another or are not parallel. At the tip of the microneedle there is another area of nanowires 7 that extend around the tip of the microneedle. These nanowires are not substantially parallel and form a fan arrangement around the tip of the microneedle but again the nanowires do not overlap one another and form a surface that in effect increases the surface area of the microneedle. An array of microneedles can include a mixture of microneedle orientations, heights and other parameters, such as whether there are nanowires present or not.

In FIG. 2e) shows the nanowires 6 covered by a membrane 8 that assists in protecting the nanowires on the microneedles as the microneedles are inserted into the skin. The membrane is usually an epoxy polyurethane type membrane which combines rigidity and porosity. However other material that has a degree of porosity to allow flow through the membrane and into the microneedles can be used.

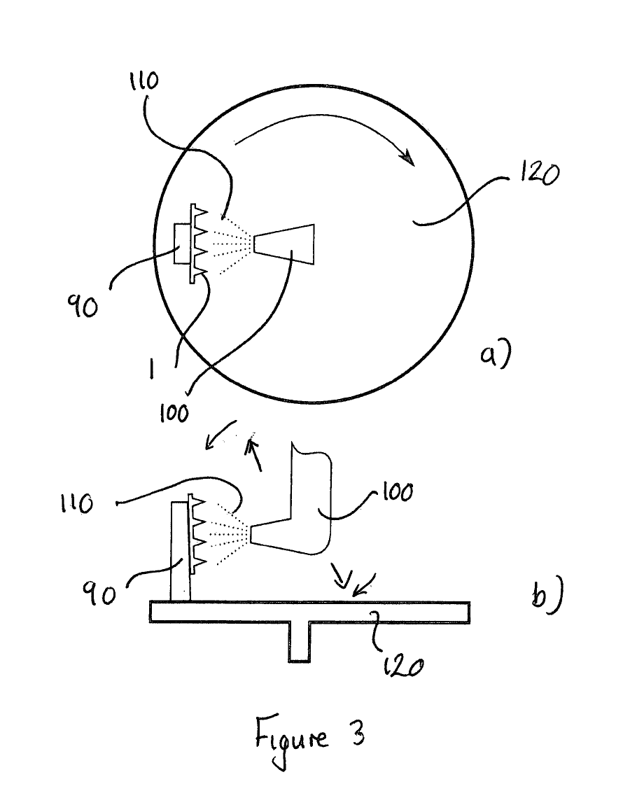

In FIG. 3 we see apparatus that is used to coat the seed layer on the microneedles of the invention. FIG. 3a shows a plan view of the apparatus and microneedles 1 are formed on a support 2 and then this whole arrangement is supported on a holder 90. The holder is placed in relation to a nozzle 100 which releases a spray 110. The holder 90 is held on a plate 120 that can rotate and about the nozzle to coat the microneedles on the holder with seed layer. The arrangement of the nozzle 100, the spinner plate 120 and the holder 90 is shown in FIG. 2a. The use of the spinner plate means that the seed layer can be laid down in a number of passes so an even layer of material is laid down on the microneedles. However it is envisaged that an arrangement may be used where the holder 90 stays in one position and this moves relative to the spray 100. The technical issue is to deposit zinc acetate to form the seed layer on the 3D structure of the needles. The samples are spin coated, with the sample being held so that the needles are horizontal and when the zinc acetate is deposited on the needle the centrifugal force applied to the needle draws the coating drawn along the length of the needle to evenly coat the needle from tip to base. If the needles are held at an angle that is a slight angle to the horizontal then buildup of material between needles is reduced. The zinc acetate is typically 0.1 M Zinc acetate in an aqueous solution and the sample is spun at 2000 to 3000 RPM for at least one minute. Once the zinc acetate is coated on the needle it is annealed at a temperature of between 150 degrees centigrade and 350 degrees centigrade for 10 minutes or more. It is also possible to coat the device with a membrane in a similar way where polymer is deposited at the base of microneedles coated with nanowires and then the device is spun so that the membrane material is drawn along the length of the microneedles/nanowires. This production method forms a membrane which is evenly distributed along the length of the microneedles/nanowires. This is just an example of how a seed layer and membrane layer can be formed on the microneedles and coating may be by other methods such as PVD deposition of the seed layer. It is also envisaged that as shown in FIG. 3(b) the head that delivers material onto the device is capable of rotation at an angle to the head to get the material to deposit on the sides of the features such as the microneedles so that material is deposited on the sides of the vertical features. What is shown is just an example of the coating method and what is important is that rotational motion is not perpendicular to the sputtering direction but at an angle. This is done with a universal joint to change the direction of the rotary motion of the substrate holder which is normally perpendicular to the sputtering direction.

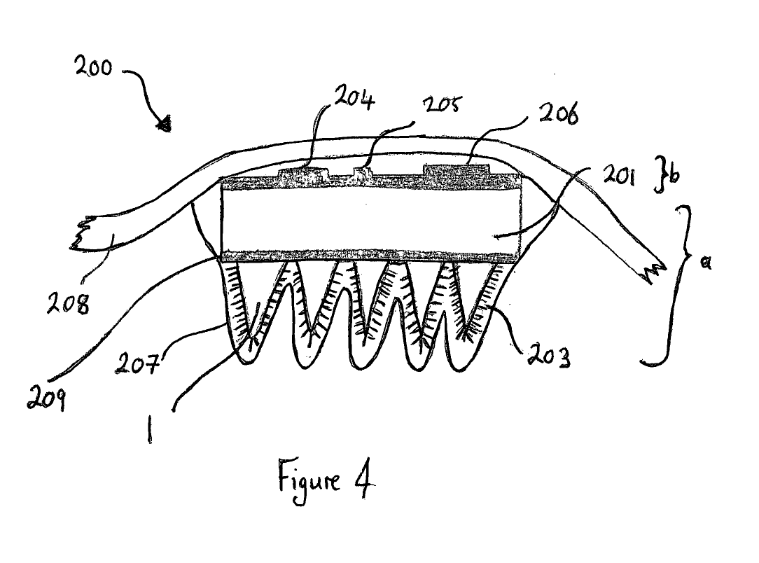

A sensor that forms part of a biological fluid monitoring device is shown generally as 200 in FIG. 4. The sensor device part of the biological fluid monitoring device includes microneedles 1 which have a number of nanowires 203 on the surface of each microneedle. The nanowires have been grown from a seed layer, for example Zinc Oxide, on the surface of the microneedles. In the diagram all of the microneedles are shown as being coated with nanowires but it may be the case that not all of the microneedles are coated with a seed layer so there are partitions in areas on the sensor to delineate areas of microneedles. These non coated areas can define barriers between areas of coated nanowires and individual areas may be functionalized with linkers for particular analytes in the biological fluid that is being monitored.

The nanowires form an increased surface area which enables the device itself to be kept as small as possible. Also having an increased surface area increases the sensitivity of the device and also allows for increase of the enzyme loading to functionalise the surface of the microneedles/nanowires. Furthermore the microneedles and nanowires are small and so this means that the sensor and associated biological fluid monitoring device can be made very small so making it less intrusive for a user. The microneedles and nanowires are covered by a membrane 207 which is porous to body fluids so the fluids can flow into the space around the microneedles/nanowires. The membrane may be porous to limit number of materials if further selection is required as to the material contacting the nanowires/microneedles.

The sensor formed of the microneedles/nanowires (which are coated with a conductive layer and then are functionalized) together with the membrane forms a first part, a) i.e. the sensor device part of a biological fluid monitoring device. The second part is the control part b) of the biological fluid monitoring device and this second part can be attached to the sensor and the second part provides the controls for the sensor. The controllers include a receiver e.g. a data recorder 204 and also a transmitter 205 which receives and forwards data from the sensor to a monitoring station and which controls and monitors the sensor periodically, transmitting information such as glucose levels and/or warnings to an external mobile device.

Furthermore, there is a built-in accelerometer 206 in the sensor electronics for the detection of movement/consciousness and fall of the user. The control part a) can be attached either permanently or releasable to the sensor part b). The whole device is then covered with an adhesive patch 208 to keep the device tight to the skin and watertight. Having parts a) and b) releasably connected to one another means that the sensor part can be discarded after a period of use and then a new sensor can be attached to the control part a). The control part can be reused by the user and data can then be collected for the life of the control part which is longer than that for the sensor part a).

The microneedles 1 typically will be 700 to 1000 μm meaning the device will be entirely painless as the microneedles are sized to avoid or minimize contact with nerve endings in the biological tissue, such as the dermis, thereby eliminating or reducing pain when the microneedles are inserted, for example into the skin. The microneedles are attached to a substrate 209 which forms the base of the microneedle(s) and this attaches the microneedle to a chip which is the interfaces with the control part a). There is no need for as bore in the microneedles as they are “bathed” in interstitial fluid.

Having the nanowires in alignment means that there is the maximum surface area available for the sensor, making it more sensitive and they are particularly useful in continuous glucose monitoring (CGM) sensors. The sensor can communicate via transmitters with external monitoring devices or may include an electronics package. The electronics package typically includes a power source (e.g., a battery), as well as electronic hardware and software for the transduction, storage, transmission, and display of measured values. The electronics package can be selectively fixed to the microneedle device, for example, so that the electronics package can be reused with a new, disposable microneedle device. The electronics package can include a mechanism for wireless or wire-based transmission of measured values to a remote device for analysis and/or display. The electronics also may include mathematical manipulation of the sensed data, for example, to average measured values or eliminate outlying datapoints so as to provide more useful measurements. This manipulation could also include prediction of trends over time. The electronics package also can include software and hardware to initiate or automate the sensing and analysis processes.

Calibration of the sensor can be accomplished using the concentration of a second analyte or the same analyte measured by another means. The primary analyte can be normalized, lowering extraction to extraction and site to site variability, by the concentration of the second analyte or same analyte from a separate measurement. Normalization may be a linear or non-linear relationship.

In a preferred embodiment for glucose sensing, a reusable sensor, which assays glucose concentration in interstitial fluid, can be calibrated daily by correlating interstitial fluid glucose values with values obtained from glucose measurements obtained from blood.

The microneedles and nanostructures which form the sensor all form one part of the device which is disposable. This part of the device can be releasably secured to the electronics of the sensor and a transmitter which can be used on a repeated basis with microneedles that have been replaced. The device will be attached to the individual by way of an adhesive patch or plaster when the microneedles, reservoir and sensor have been secured to the sensor electronics and transmitter.

The devices disclosed herein form minimally invasive bioassay devices and are also useful in the transport of biological fluids from within or across a variety of biological barriers including the skin (or parts thereof); the blood-brain barrier; mucosal tissue; blood vessels; lymphatic vessels; cell membranes; epithelial tissue; and endothelial tissue. The biological barriers can be in humans or other types of animals, as well as in plants, insects, or other organisms, including bacteria, yeast, fungi, and embryos. In preferred embodiments, biological fluids are withdrawn from skin, more preferably human skin, for minimally invasive diagnostic sensing. Biological fluids useful with the devices described herein include blood, lymph, interstitial fluid, and intracellular fluid. In a preferred embodiment, the biological fluid to be withdrawn or sensed is interstitial fluid.

A variety of analytes are routinely measured in the blood, lymph or other body fluids. Examples of typical analytes that can be measured include blood sugar (glucose), cholesterol, bilirubin, creatine, various metabolic enzymes, hemoglobin, heparin, hematocrit, vitamin K or other clotting factors, uric acid, carcinoembryonic antigen or other tumor antigens, and various reproductive hormones such as those associated with ovulation or pregnancy. Other substances or properties that would be desirable to detect include lactate (important for athletes), oxygen, pH, alcohol, tobacco metabolites, and illegal drugs (important for both medical diagnosis and law enforcement). With the use of an appropriate receptor at the surface of the nanostructures, the technology can be applied to monitor other chronic diseases, such as heart diseases, stroke, chronic obstructive pulmonary diseases and asthma etc.

It is to be understood that the above embodiments have been provided only by way of exemplification of this invention, such as those detailed below, and that further modifications and improvements thereto, as would be apparent to persons skilled in the relevant art, are deemed to fall within the broad scope and ambit of the present invention described. Furthermore where individual embodiments are discussed, the invention is intended to cover combinations of those embodiments as well.

1. A microneedle device for use in a sensor, said microneedle device including at least one microneedle having one or more nanowires on a surface of said at least one microneedle.

2-31. (canceled)
